(19) United States
(12) Patent Application Publication
(10) Pub. No.: US 2007/0184095 A1
(43) Pub. Date: Aug. 9, 2007

(54) OSMIUM COMPOUNDS FOR THE TREATMENT OF PSORIASIS

(76) Inventor: Adam Heller, Austin, TX (US)

Correspondence Address: HELLER EHRMAN LLP, 275 MIDDLEFIELD ROAD, MENLO PARK, CA, 94025-3506, US

(21) Appl. No.: 11/669,753

(22) Filed: Jan. 31, 2007

Related U.S. Application Data
(60) Provisional application No. 60/764,289, filed on Jan. 31, 2006.
(60) Provisional application No. 60/830,595, filed on Jul. 12, 2006.

(51) Int. Cl. A61K 33/24

(52) U.S. Cl. 424/445; 424/617

(57) ABSTRACT

Pharmaceutically acceptable compositions and methods for the treatment of psoriasis are disclosed. The compositions contain osmium compounds. The nominal valence of the osmium in the preferred compounds is four or greater, and is less than eight. The atoms proximal to the osmium atoms include oxygen atoms, or functions exchanged by water under physiological conditions. The preferred osmium compounds have three or more oxygen atoms and/or water exchangeable functions proximal to their osmium atoms. Psoriasis is treated by exposing the affected skin to osmium tetroxide vapor, or by topically applying a solution or paste of the osmium containing composition.

### RELATED APPLICATIONS

The present application claims the benefit of the filing dates of U.S. Provisional Patent Application No. 60/764,289 filed on Jan. 31, 2006 and U.S. Provisional Patent Application No. 60/830,595 filed on Jul. 12, 2006, the entire disclosures of which are hereby expressly incorporated by reference.

This application is further related to U.S. Patent Application Publication No. 2005/0025805 A1, published on Feb. 3, 2005, U.S. Patent Application Publication No. 2005/0025804 A1, published on Feb. 3, 2005, and U.S. Provisional Patent Applications 60/490,767, filed on Jul. 28, 2003; 60/503,200, filed on Sep. 15, 2003; and 60/539,695, filed on Jan. 27, 2004, the full disclosures of which are hereby expressly incorporated by reference.

### FIELD OF THE INVENTION

The invention relates to topically applied pharmaceutically acceptable compositions containing osmium compounds for treatment of psoriasis, and methods of treating psoriasis with topically applied pharmaceutically acceptable compositions containing osmium compounds are disclosed.

### BACKGROUND OF THE INVENTION

Prevalence of Psoriasis

According to the US National Institutes of Health Medical Encyclopedia, website http://www.nlm.nih.gov/medlineplus/ency/article/000434.htm, psoriasis affects about 2.7% of the people of the world. In the United States, about 3 million people show symptoms of psoriasis at any given time. Psoriasis may affect any or all parts of the skin, but it is more commonly seen on the skin of the trunk, elbows, knees and/or scalp, on skin folds, or in the fingernails and/or toenails. Psoriasis may be aggravated by injury or irritation, such as cuts, burns, rashes or insect bites. It is particularly severe in immunosuppressed people, like those with AIDS or undergoing chemotherapy for cancer, and in people who have other autoimmune disorders, such as rheumatoid arthritis. In psoriatic arthritis, both a joint and the skin are affected.

Symptoms of Psoriasis

When the skin is healthy, it takes about a month for new skin cells to move up from the lower layers to the surface of the skin. In psoriasis, this process takes only a few days, and it results in the build-up of dead skin cells and formation of thick scales.

Keratinocyte proliferation is characteristic of psoriasis. Symptoms of psoriasis include, for example, patches of skin that can (a) be dry and/or red; and/or (b) be covered with silvery scales; and/or (c) be raised; and/or (d) have red borders; and/or (e) crack and/or become painful; and/or (f) be discrete and/or demarcated. Additional symptoms may include, for example, (a) skin lesions, such as pustules; and/or (b) cracking of skin; and/or (c) skin redness and/or inflammation; and/or (d) itching; and/or (e) small scaling dots on the skin, especially in children; and/or (f) joint pain or aching, which may be associated with psoriatic arthritis. Further abnormalities in psoriasis may include, for example, nail abnormalities; genital lesions in males; and burning, itching, discharge or increased tearing of the eye.

Current View of Psoriasis

Psoriasis is considered to be an immune disease. It is classified in many recent publications as an autoimmune disease, a class of diseases in which the immune system targets the body's own cells. Publications suggest that psoriasis is a type 1 autoimmune disease, mediated, for example, by interferon gamma and/or other inflammatory cytokines, and/or by T-lymphocytes. For example, IFN-gamma-producing CD4+Th1-lymphocytes are considered to be of importance in the pathogenesis of psoriasis, as they influence differentiation and functioning of antigen presenting cells, mast cells, neutrophils and endothelial cells. The inflammatory cascade provokes neo-angiogenesis in the dermis and proliferation of keratinocytes. Lowes et al. recently reported that CD11c+ cells with markers of dendritic cells are a major cell type in the skin lesions of psoriasis. These CD11c+ cells, which are evident in both epidermis and dermis, are sites for expression of two mediators of inflammation in diseased skin, inducible nitric oxide synthase (iNOS) and TNF-α. These cells also express HLA-DR, CD40, and CD86 and the dendritic cell maturation markers DCLAMP and CD83.

Current Treatments of Psoriasis

Mild psoriasis is now treated with non-steroidal anti-inflammatory drugs (NSAIDs), exemplified by topically applied salicylic acid and its orally taken derivative, aspirin (known to inhibit NF-κB); topically applied coal tar; orally taken vitamin D derivatives, like calcipotriol; UV-B phototherapy; and topically applied glucocorticosteroids, like betametasone, known to down-regulate CCL27. Combinations of these are often used. Traditional treatments of severe psoriasis include systemic, orally taken, disease-modifying anti-rheumatic immunosuppressive drugs (DMARDs), like methotrexate, cyclosporin, psoralen plus UVA (PUVA), oral retinoids and fumaric acid esters, gold salts and leflunomide. More recently, biological drugs were introduced to treat severe psoriasis. These include (a) T-cell count lowering AMEMIVE® (alefacept), a recombinant protein binding to CD2 on memory-effector T lymphocytes, inhibiting their activation and reducing the number of these cells. It is a fusion protein composed of leukocyte function-associated antigen type 3 (LFA-3) protein and human IgG1 Fc domains, systemically administered by intramuscular injection. (b) RAPTIVA® (efalizumab), which is a humanized monoclonal antibody against the CD11a subunit of leukocyte function-associated antigen-1 (LFA-1). CD11a is a T-cell surface molecule, important in T-cell activation, T-cell migration into skin, and cytotoxic T-cell function. RAPTIVA® (efalizumab) binds to the CD11a on T-cells and reversibly blocks the interaction between LFA-1 and its adhesion partner molecule ICAM-1. Weekly systemic injections of RAPTIVA® (efalizumab) must continue indefinitely to maintain improvement. (c) ENBREL® (etanercept), a human TNF-α receptor, made by fusing two natural TNF-receptors. Its affinity for TNF-α is greater than that of the natural monomeric TNF-α receptor of the immune system. ENBREL® (etanercept) is systemically administered, and deactivates TNF-α upon binding. (d) HUMIRA® (adalimumab), a human IgG1 monoclonal TNF-α-binding and inactivating antibody, is used for treating psoriatic arthritis. Unlike the other TNF-α inhibitors, it is locally injected. (e) REMICADE® (infliximab), a chimeric (mouse-human) IgG1 monoclonal antibody, which binds to and inactivates TNF-α, and administered by systemic injection.

The need for a safe, less expensive, topically applied drug for psoriasis management. The biological drugs ameliorate the symptoms of, but do not cure, psoriasis. All five biological drugs listed above are injected, and the injections must continue indefinitely. Topically applied compositions are needed, as these could be safer than the injected or otherwise systemically, e.g. orally, administered drugs, injected and otherwise systemically administered drugs being more likely to affect also organs other than the targeted psoriatic skin. There is also a need to reduce the heavy financial burden associated with treating psoriasis. The annual cost of treating psoriasis with any of the five biological drugs in the USA is between about $ 15,000 and about $ 20,000, an amount representing about half of the annual income of many U.S. wage earners. The price of cyclosporine is also high, the drug costing annually about $ 10,000.

Although the non-biological drug cyclosporine and the biological drugs are generally safe at their dermatological dosage, side effects have been reported. Cyclosporin increases the risk of squamous cell carcinoma of the skin. Adalimumab increases the incidence of serious infections by two-fold, its most notable complication being reactivation of tuberculosis. Among the infliximab treated patients a small percentage reported pneumonia, tuberculosis, lymphoma, drug-induced lupus and hepatotoxicity. Antiefalizumab antibodies developed in approximately 5% of the subjects who were treated with efalizumab. Immune-mediated thrombocytopenia platelet counts at or below 52,000 cells/microliter have been observed in 0.3% of the efalizumab treated patients and four patients developed hemolytic anaemia. The overall incidence of hospitalization for infections was 1.6 per 100 patient-years for efalizumab-treated patients compared with 1.2 per 100 patient-years for placebo-treated patients.

### SUMMARY OF THE INVENTION

Pharmaceutically acceptable compositions containing an osmium compound for treating psoriasis are disclosed. A particular group of the osmium compounds used in the compositions and methods of the present invention catalyze the dismutation of the superoxide radical anion O2.- to O2 and H2O2. The compositions are topically applied to the skin. They may be immobilized on and/or near the skin for rapid or slow release and/or for controlled release. The compositions may, for example, be aqueous solutions of osmium compounds or difficult to oxidize, or stabilized, oil based or organic solutions of osmium compounds. Methods of treating psoriasis by topically applying these solutions or by exposing the psoriasis affected skin to a gas containing an osmium compound, such as osmium tetroxide, OsO4, are also disclosed and form part of the invention.

### TERMS AND DEFINITIONS

Skin. Skin means the air-contacting part of the human body to a depth of about 7 mm from the air interface; as such, it also includes the nails.

Pharmaceutically acceptable means that the topically applied composition or dressing is non-toxic when applied to the skin at the recommended dosage and suitable for use for the treatment of humans and animals. Such pharmaceutically acceptable compositions are free of materials that are incompatible with such use.

Topically applied means that the ointment, cream, emollient, balm, lotion, solution, salve, unguent, or any other pharmaceutical form is applied to some or all of that portion of the patient's skin that is, or has been, affected by, or shows, or has shown, one or more symptoms of psoriasis. Topical composition means an ointment, cream, emollient, balm, lotion, solution, salve, unguent, or any other pharmaceutical form intended for topical application to the skin of a patient showing any of the symptoms of psoriasis.

Osmium compound means any compound containing osmium used for treating psoriasis. The nominal valence of osmium in the preferred pharmaceutically useful osmium compounds is at least four, more preferably is at least five, and most preferably is at least six. In general, the atoms proximal to the osmium atom of the compound include at least three oxygen atoms, or precursors of compounds where the atoms proximal to the osmium atom of the compound include at least three oxygen atoms. Preferably, the atoms proximal to the osmium atom of the compound include at least four oxygen atoms, or precursors of compounds where the atoms proximal to the osmium atom of the compound include at least four oxygen atoms.

Matrix means the part of an unguent, paste, ointment, lotion, cream, salve, solution, or gel applied to the skin in which the osmium compound is dissolved or dispersed.

Water-based means that the continuous phase of the matrix is mostly aqueous.

Oil-based matrix has the same meaning as organic matrix. Both mean that the continuous phase of the matrix is mostly non-aqueous and is, generally, rich in carbon.

Stabilized means containing an oxidant that slows the reduction of osmium tetroxide, usually by re-oxidizing osmium compounds in which the valence of osmium is less than eight. Osmium tetroxide solutions can be stabilized, for example, with hydrogen peroxide or ammonium persulfate, with a peracid, or with a salt of a peracid, such as ammonium peroxydisulfate, or with a poly(vinylpyridine-N-oxide), such as poly(2-vinylpyridine-N-oxide), or poly(4-vinylpyridine-N-oxide), or a derivative of a poly(vinylpyridine-N-oxide).

Non oxidizable refers to a matrix that is not as rapidly oxidized by OsO4 dissolved in it at 25° C. as are, for example, aliphatic alcohols, aliphatic ketones, aliphatic esters, alkenes or phenols. Exemplary non oxidizable matrices include siloxanes, like polymeric poly(dimethylsiloxane) based oils or greases and fluorinated liquids, greases or waxes, exemplified by those sold under the trade name Krytox by E. I. DuPont de Nemours & Co. of Wilmington, Del.

Stabilized solution refers to an organic solvent or oil based solution, usually of OsO4, to which a second oxidant, such as hydrogen peroxide, or ammonium peroxydisulfate or ammonium peroxydisulfate, or a poly(N-vinylpyridine-N-oxide) has been added. The second or added oxidant is referred to as the stabilizer.

Dressing means a covering for a wound or surgical site, typically composed of a cloth, fabric, synthetic membrane, gauze, or the like. It is usually a polymer-containing matrix covering an area of the skin. The dressing may or may not be in intimate contact with the skin. It can be, for example, a cloth or gauze, or it can be a polymer solution painted or sprayed on the skin, the polymer solidifying on the skin when the solvent dries off and/or when the polymer crosslinks. Dressings also include gels, typically cross-linked hydrogels, which are intended principally to cover and protect wounds, surgical sites, and the like.

Adverse inflammation is an inflammation other than inflammation resulting of infection by a pathogen, such as a virus, bacterium, fungus or parasite.

Controlled release means release of the active compound or its precursor so that it contacts, and is available for absorption by, the skin over a period of time. The time period is generally longer than about 1 min and is shorter than 2 weeks. The period is preferably longer than about one hour and is shorter than about 3 days.

O2.- dismutation catalyst and dismutation catalyst both mean a catalyst of one or both of the O2.- consuming reactions 2 O2.-+2 H+→O2+H2O2 and 2 O2.-+H+→O2+OOH— the O2.-.

Water-exchangeable means an ion or molecule in the inner coordination sphere of an osmium ion center that can be exchanged by a molecule of water or by a hydroxide anion.

### DETAILED DESCRIPTION OF THE INVENTION

Nitric oxide and the superoxide radical anion in psoriasis. Psoriasis is an adverse inflammation of the skin. In psoriasis, one or more of the immune system's signaling molecules trigger events leading to a local excess of nitric oxide, .NO. The excess NO is produced by the inducible nitric oxide synthase, iNOS-catalyzed oxidation of arginine by O2. iNOS is expressed or over-expressed by some of the immune system's cells, and by skin cells stimulated by inflammatory chemicals. Signaling molecules, released by skin cells altered or damaged by cytotoxic species formed of, or associated with, the local .NO excess, may recruit more of the iNOS expressing or over-expressing cells. Thus, in psoriasis, an amplified skin cell-altering or damaging feedback loop may result.

The adverse role of NO in psoriasis is, at least in part, a result of its combining with the superoxide anion radical, O2.-. The radical anion O2.- is generated mostly in the mitochondria of the skin cells, and is also generated by leucocytes, like neutrophils or macrophages. The product of the combination of .NO and O2.- is the peroxynitrite anion, ONOO—, which is more cytotoxic than the combining reactants. The daughter products of ONOO— are even more cytotoxic. Part of the ONOO— is protonated, in a pH dependent equilibrium reaction, to peroxynitrous acid, ONOOH. ONOOH decomposes in part to the highly cytotoxic hydroxyl radical, OH, and to also cytotoxic nitrogen dioxide, .NO2. Part of the ONOO— combines with cytosolic carbon dioxide, CO2, to form ONOOCO2-, which decomposes in part to the highly cytotoxic carbonate radical anion, CO3- and to .NO2. The .NO2, formed by either reaction, is known to react with protein or peptide tyrosine residues to produce 3-nitrotyrosine residues. By nitrating and/or nitrosylating tyrosine residues, it changes biological signaling pathways, such as enzyme-involving, for example kinase or oxidase-involving, and/or receptor-involving pathways.

iNOS amplifying feedback loops of psoriasis. CD11c+ cells, with markers of dendritic cells, are major constituents in psoriatic lesions. These cells, found in the epidermis and in the dermis, express, among others, iNOS and tumor necrosis factor α, TNF-α iNOS and TNF-α are amplifiers of inflammatory feedback loops. In one of the possible primary iNOS amplifying feedback loops of psoriasis, iNOS binds cyclooxygenase-2 (COX-2). The .NO generated in the iNOS-COX-2 complex nitrosylates the COX-2, and the nitrosylation increases the activity of the COX-2. Because COX-2 catalyzes the cyclooxidation of arachidonic acid to prostaglandin-E2 (PGE2), the nitrosylation raises the local concentration of PGE2. PGE2 enhances the chemotactic response of monocytes to monocyte-chemoattractant protein-1 (MCP-1) It causes recruitment of more monocytes to the already adversely inflamed site, the recruited monocytes maturing to iNOS expressing macrophages, further increasing the local iNOS activity, the increased iNOS activity resulting in even greater local .NO generation. Furthermore, the greater local iNOS concentration results in even more complexing of COX-2, which further increases the local PGE2 concentration. In a second amplifying sub-loop of this primary loop, PGE2 enhances in mast cells the secretion of MCP-1, effecting further monocyte recruitment.

In a related, also amplifying, loop of psoriasis, proliferating psoriatic keratinocytes express MCP-1 when stimulated by TNF-α or by interferon-γ, IFN-γ, two cytokines produced by the CD11c+ cells of the psoriatic lesions. Again, more monocytes are recruited, more iNOS is expressed, more cells are damaged, and more keratinocytes, producing even more MCP-1, recruit even more monocytes to the psoriatic lesion. The monocytes maturing to macrophages, express even more iNOS.

In yet another type of possible primary feedback loop of psoriasis, the local iNOS activity is amplified by leakage of cytokines, particularly TNF-α, from the damaged cells; the leaked TNF-α molecules stimulate the expression of MCP-1 by the keratinocytes; the chemokine receptor CCR2, expressed on the circulating monocytes of patients with psoriasis, binds the keratinocyte-expressed MCP-1, causing the recruitment of even more monocytes. Again, the monocytes maturing to macrophages, express even more iNOS.

Relatedly, induction of activation of STAT-3 and NF-kappa B is suppressed by overexpression of Mn-superoxide dismutase and activated Stat3 is characteristic of epidermal keratinocytes in psoriatic lesions.

Elevated concentrations of O2.- and O2.--amplifying feedback loops in psoriasis. Sedgwick, Bergstresser and Hurd reported that sera from patients with psoriasis induced O2.- generation when incubated with normal polymorphonuclear leukocytes and zymosan. Elevated concentrations of O2. - in the psoriatic lesions have also been reported. That O2.- has a role in the pathogenesis of psoriasis was seen in cell cultures, where up-regulation of Cu—Zn superoxide dismutase inhibited keratinocyte proliferation. Mn-superoxide dismutase is known to be inactivated by peroxynitrite, ONOO—. Thus, in a possible primary feedback loop underlying psoriasis, Mn-superoxide dismutase is inactivated by the ONOO— produced in the reaction of NO with O2.-, whereupon the local concentration of O2.- is increased, the fraction of the available .NO reacting with O2.- rather than being oxidized to nitrate or nitrite is increased, and because even more ONOO— is produced, which inactivates even more the one or both of the Mn-superoxide dismutase.

Treatment of psoriasis with topically applied osmium compounds. To alleviate the adverse inflammation of psoriasis, it is desired to decrease the concentration of O2.- in the psoriasis affected part of the skin, also referred to as the psoriatic lesion. As disclosed in Application 2005/0025805 (A1) and in a paper of Goldstein, Czapski and the Applicant, OsO4 and other osmium compounds are potent O2.- dismutation catalysts, decreasing the concentration of O2.-. Because the molecular mass of the fastest O2.- dismutating enzyme, believed to be Cu,Zn-superoxide dismutase, is 32-kDa and because the molecular mass of OsO4 is only 254 Da, and because the catalytic rate constant of OsO4 in O2.- dismutation is high, about (1.43±0.04)×109 M−1s−1, the specific activity of OsO4, meaning its catalytic activity per unit mass, is about 60 times greater than that of Cu,Zn-superoxide dismutase.

OsO4 may permeate through the skin, through cell membranes and through the mitochondrial membrane. OsO4 may readily pass biological lipid bilayer membranes, because it is soluble both in water and in non-polar solvents, like carbon tetrachloride, hexane, and liquid poly(dimethylsiloxane). It may thus rapidly permeate through cell membranes and membranes of organelles in cells, such as mitochondria, where most of the O2.- is produced and where the O2.- concentration is highest. Mitochondrial change or damage, that may underlie psoriasis, may be slowed or stopped by the pharmaceutically acceptable osmium compounds. Thus OsO4 and/or precursors yielding OsO4 upon reaction with O2.- are preferred active components of the pharmaceutically useful compositions for the topical treatment of psoriasis.

Reductive OsO4 immobilization in cells and in their mitochondria, and recovery of OsO4 when needed, by either peroxide, for example hydrogen peroxide or ammonium persulfate or ammonium persulfate, oxidation, and/or O2.- -oxidation, of immobilized OsVI and lower oxidation state osmium compounds. The OsO4 may permeate rapidly through membranes, such as those of the psoriatic skin cells, and of their mitochondria, and may be reduced in the cells and/or in their mitochondria, initially to immobile, nominally six-valent, long lived, osmium compounds, denoted as OsVI. Thus after entering the psoriatic cells and/or their mitochondria the OsO4 may be reductively immobilized and/or stored as an OsVI compound. The stored and trapped OsVI compound may then be oxidatively re-activated, during flare up of psoriasis when the when the O2.- concentration is high, by reacting with O2.-, to re-form the catalytically most active, nominally hepta/octa-valent, OsVII/VIII, redox couple, of which OsO4 is part, and which is about as active a dismutation catalyst as OsO4 itself. The O2.- may regenerate, particularly in the mitochondria where it is most needed, the active dismutation catalyst during the flare-up of psoriasis. Thus, after the OsO4 is reductively immobilized in the psoriatic skin, it may limit the upper O2.- concentration and reduce or stop the amplification in at least some of the amplifying cycles underlying psoriasis, such as those involving in one of their steps nitration or nitrosylation of tyrosine residues, some of which were described in paragraphs [0025] and/or [0026]. Not only is the specific activity for O2.- dismutation of OsO4, the OsVIII -member of the OsVII/VIII redox couple high, but has the additional advantage of not being inactivated by ONOO—, which abounds in the flare-up of psoriasis and which is known to inactivate natural Mn-superoxide dismutase. For these reasons, and because neither OsVI nor OsO4 is particularly toxic, both OsO4 and those osmium compounds in which the nominal valence of osmium is four, OsIV, and/or five, OsV, and/or or six, OsVI, and which are oxidized by O2.- to osmium compounds in which the nominal valence of the osmium in the compound is seven OsVII, such as the OsO4- anion and/or eight, as it is in OsO4, are particularly useful in topically treating psoriasis.

The useful compositions for treating psoriasis contain platinum group element compounds, osmium compounds being preferred. The pharmaceutically useful compositions for treating psoriasis are generally compounds of the group VIII elements, preferably of the platinum group elements. Their oxygen-containing compounds are preferred. Osmium compounds in which the formal or nominal valence of osmium is greater than four are particularly preferred for use in the pharmaceutically acceptable compositions for topically treating psoriasis. Compounds in which the formal or nominal valence of osmium is greater than six are more preferred and OsO4 is most preferred. Because compounds in which osmium is tetra-valent (OsV), penta-valent (OsV), hexa-valent (OsVI), or hepta-valent (OsVII) areoxidized by O2.- to the catalytically uniquely active OsVIII/OsVII redox couple, they are useful. In general, OsVII compounds are preferred over OsVI compounds; OsVI compounds are preferred over OsV compounds; and OsV compounds are preferred over OsV compounds. Useful exemplary OsVI compounds are produced by reactions of sugars, such as glucose, with OsO4, or by reacting OsO4 with compounds having heterocyclic unsaturated rings, like pyridine or 2,2′-bipyridine.

Osmium compounds in which the Os is bound to at least three oxygens are preferred, and those where osmium is bound to at least four oxygen atoms are more preferred. Osmium compounds or complexes, hydrolyzed under physiological conditions such that after hydrolysis the osmium is linked to at least three oxygen atoms, more preferably four oxygen atoms, are useful in treating psoriasis. Physiological conditions are those of an about pH 7.3 aqueous 0.14 M NaCl, 0.02 phosphate-solution at about 37° C. In the complexes of osmium, the water-exchanged ligands can be, for example, halide anions, ammonia, N-oxides, phosphine-oxides, or sulfoxides.

The bonds between the osmium and the proximal oxygen atom can be electrostatic, also termed ionic, and/or covalent, and/or coordinative. Exemplary useful osmium compounds include salts like K2OsO4, where the formal or nominal oxidation state of osmium is (VI), or a polymer, such as poly-2-vinylpyridine or poly-4-vinylpyridine reacted OsO4, where the formal oxidation state of osmium is also (VI); and salts like (NH4)2OsO3, where the formal oxidation state of osmium is (IV). The osmium compound or salt can be a non-stoichiometric composition, or a composition in which the osmium is of mixed-valence. The solubility of the useful osmium compounds in water at 37° C. is generally greater than about 10-9 M, and compounds with a solubility exceeding about 10-8 M are preferred.

The preferred matrices. OsO4, which is soluble both in organic solvents and in water, and is volatile at ambient temperature, can be released in a controlled manner from either an organic solvent or an oil based matrix, or a water based matrix, applied to the skin, or from a dressing on or near the skin. Solutions of osmium compounds are generally preferred over their suspensions or emulsions. OsO4 also dissolves in and is stable in water. Aqueous solutions in which the concentration of OsO4 is between about 0.05 weight % and about 10 weight % are preferred, and those in which its concentration is between about 0.1 and about 1 weight % are most preferred.

The aqueous solutions with which the OsO4 is diluted may contain pharmaceutically acceptable organic additives reducing the OsO4 to a colored lower-valent, preferably six-valent osmium compound. The additive can be, for example, a sugar, such as glucose, added in an amount equal to or greater than a 1:1 molar ratio. Such a solution is convenient to store. Some or all of the OsO4 can be regenerated prior to application by adding an oxidant, for example hydrogen peroxide or ammonium persulfate or ammonium persulfate. Alternatively, stabilized solutions of OsO4 in pharmaceutically acceptable organic liquids, gels, pastes or greases can be used. These may contain, for example, siloxane functions and can be optionally stabilized by a stabilizer, such as hydrogen peroxide or ammonium persulfate or ammonium persulfate.

The OsO4 can be incorporated in dressings over the skin, optionally, but not necessarily, in direct contact with the skin. Either the water or the oil-based osmium compound containing matrices can be incorporated in a dressing. When the osmium compound is a cation or anion, it can be stored in, and slowly released from, an ion exchange resin, or from a polycationic, or polyanionic, water-based hydrogel, which can be applied to the skin, or be part of a dressing applied to the skin. It can be released, for example, from a hydrogel, or a lotion, or a salve, or an ointment or a cream, or a dressing applied to the skin.

Treatment by exposure of the psoriatic part of the skin to OsO4 vapor. Because the vapor pressure of OsO4 is substantial at ambient, as well as typical skin temperatures, any method providing for exposure of the psoriatic part of the skin to its vapor is appropriate.

The psoriatic part of the skin can be exposed to gas phase OsO4 over pure solid OsO4, or pure liquid OsO4, or over a solution of OsO4, the gaseous OsO4 being in a container. The OsO4 exposed surface of the container is made preferably of a material which is not oxidized by OsO4, such as an inorganic glass, like a silicate glass, or of Teflon, or of a metal like titanium or aluminum, the surface of which is pre-oxidized, for example by anodization, by heating in air, or exposure to a gaseous O2-containing plasma. Alternatively, the OsO4 can be released from a dressing, which can be optionally conformal and in contact with the skin.

The preferred concentration range of the osmium compound in the matrix. The preferred concentration of the osmium compounds in water, such as that of the exemplary OsO4, is typically between about 0.05 weight % and about 10 weight %, more preferably between about 0.05 weight % and about 5 weight %, and most preferably between about 0.1 weight % and about 1 weight %. In the organic matrices the concentration of the osmium compounds is typically greater than about 0.03 weight % and less than about 10 weight %, the preferred concentration being greater than about 0.05 weight % and about 2 weight %.

While the above is a complete description of the preferred embodiments of the invention, various alternatives, modifications, and equivalents may be used. Therefore, the above description should not be taken as limiting the scope of the invention which is defined by the appended claims.

### EXAMPLES

### Example 1

OsO4 solutions. Crystalline, pure OsO4 sealed in a glass ampoule Catalog Number 05500, was purchased from Sigma Aldrich, St. Louis, Mo. The crystalline OsO4 was dissolved in de-ionized water under a hood and diluted to 0.1 wt. %; 0.23 wt %; 0.5 wt % and 1 wt % OsO4 concentrations in water. All solutions were stored in gas-tight glass vials with Teflon™ caps.

### Example 2

Absence of persistent change in the healthy skin. An aqueous 1.0% by weight OsO4 solution was applied to the skin on a healthy male volunteer using a surgical gauze saturated with the solution, in a breeze-vented room with open widows to avoid excessive inhalation of OsO4. Before applying the aqueous OsO4 solution, the lower arm and elbow were washed with soap water, and towel dried in order to make the subsequently applied aqueous OsO4 spread about evenly on the skin that became more hydrophilic as a result of the wash. About 5 mL the 1.0% by weight aqueous OsO4 solution were applied to about 50 cm2 of the skin of the lower arm, near the elbow, using a glass dropper, spreading the solution about evenly with the dropper. The area was then covered with non-adhering surgical gauze secured on the treated skin. The treated area was then wrapped with multiple layers of Saran wrap, in order to reduce the evaporative loss of OsO4. Saran film, was used because the permeability of water, gases and other organic and inorganic compounds is low in Saran films. The low permeability Saran film consisted mostly of polyvinylidene chloride. The skin treated with the OsO4 solution was kept wrapped for about 6 hours, to allow the OsO4 to permeate through the skin. Subsequently, the dressing was discarded. Penetration of the skin by the OsO4 was evident from the dark, nearly black, coloration of the skin, resulting from reduction of the colorless OsO4 to colored, lower-valent, osmium compounds. The color of the skin was persistent: it did not wash off and could not be rubbed off. The black color started to visibly fade after 3 days, and after 5 days the fading was most evident. Progressive fading was observed till day 14, when there was only a trace of black coloring. Coloring of the skin was no longer discernible, even upon close inspection, after 3 weeks. There was no scarring, hair loss or other change in appearance of the skin. The subject treated did not experience itching or pain of the skin or of any part of his arm either during the 3 week test or at any time after the test.

### Example 3

Alleviation of symptoms of persistent psoriasis. A 76 year old female subject who suffered from severe psoriasis of the skin above the ankle for more than 5 years was treated. After obtaining her informed consent, her approximately 120 cm2 psoriatic lesion was treated as follows. After washing the psoriatic skin with soap and warm water and drying it with a towel, 10 mL of a 0.25% by weight aqueous OsO4 solution was spread on the most severely psoriasis affected skin, the area of which was about 120 cm2, using a soft toothbrush. The skin to which the solution was applied was then wrapper in about 5 layers of Saran wrap. The subject was instructed to keep the Saran wrap on overnight, for about 10 hours. The subject was treated once and for about a month after the treatment there was little or no improvement. About one month after the single treatment, the patient reported gradual improvement that became pronounced after about six weeks, when the swelling and the intense red color, characteristic of psoriasis subsided.

### Example 4

A psoriatic lesion of the skin is treated as described in Example 3, except that the solution applied is a 0.5% by weight solution.

### Example 5

A psoriatic lesion of the skin is treated as described in Example 3, except that the solution applied is a 0.5% by weight solution, and treatment is repeated bi-monthly (6 times a year).

### Example 6

A psoriatic lesion of the skin is treated as described in Example 3, except that the solution applied is a 1% by weight solution and treatment is repeated twice a year.

### Example 7

Treatment by exposure of the psoriatic part of the skin to OsO4 vapor. The psoriatic part of the skin is exposed to vapor phase OsO4 over pure OsO4. The OsO4 exposed surface of the container is made of a material which is not oxidized by OsO4, such as silicate glass or Teflon, or of a metal like titanium or aluminum, the surface of which is pre-oxidized, for example by anodization, by heating in air, or exposure to a gaseous O2-containing plasma.

### Example 8

Crystalline, pure OsO4 sealed in a glass ampoule Catalog Number 05500, is purchased from Sigma Aldrich, St. Louis, Mo. The crystalline OsO4 is dissolved in de-ionized water under a hood and diluted to 1 wt. % OsO4 concentrations with a 0.5 M aqueous glucose solution and stored in gas-tight glass vials with a Teflon™ cap. After storage for at least one month at room temperature, the dark suspension, in which the osmium is glucose-reacted and mostly six valent, and are mixed with an equal volume of 1% by weight hydrogen peroxide or ammonium persulfate in deionized water and stored for about 12 hours. The hydrogen peroxide or ammonium persulfate is expected to re-oxidize in part the osmium to osmium tetroxide. The rejuvenated solution is applied to the skin according to the protocol of Example 3.

### Example 9

Crystalline, pure OsO4 sealed in a glass ampoule Catalog Number 05500, is purchased from Sigma Aldrich, St. Louis, Mo. The crystalline OsO4 is dissolved in de-ionized water under a hood and diluted to 1 wt. % OsO4 concentrations with a 0.5 M aqueous glucose solution and stored in gas-tight glass vials with a Teflon™ cap. After storage for at least one month at room temperature, the dark suspension, in which the osmium is mostly glucose-reacted six valent is mixed with an equal volume of 3% by weight ammonium persulfate in deionized water and stored for about 12 hours. The ammonium persulfate is expected to re-oxidize in part the osmium to osmium tetroxide. The rejuvenated solution is applied to the skin according to the protocol of Example 3.

### REFERENCES

1. Lowes, M. A., et al., Increase in TNF-{alpha} and inducible nitric oxide synthase-expressing dendritic cells in psoriasis and reduction with efalizumab (anti-CD11a). Proc Natl Acad Sci USA, 2005. 102(52): p. 19057-62.
2. Gordon, K. B., et al., The tumour necrosis factor-alpha inhibitor adalimumab rapidly reverses the decrease in epidermal Langerhans cell density in psoriatic plaques. Br J Dermatol, 2005. 153(5): p. 945-53.
3. Reich, K., et al., Infliximab induction and maintenance therapy for moderate-to-severe psoriasis: a phase III, multicentre, double-blind trial. Lancet, 2005. 366(9494): p. 1367-74.
4. Brandt, J. and J. Braun, Anti-TNF-alpha agents in the treatment of psoriatic arthritis. Expert Opin Biol Ther, 2006. 6(2): p. 99-107.
5. Behnam, S. M., S. E. Behnam, and J. Y. Koo, Review of cyclosporine immunosuppressive safety data in dermatology patients after two decades of use. J Drugs Dermatol, 2005. 4(2): p. 189-94.
6. Berth-Jones, J., The use of ciclosporin in psoriasis. J Dermatolog Treat, 2005. 16(5-6): p. 258-77.
7. Scheinfeld, N., Adalimumab: a review of side effects. Expert Opin Drug Saf, 2005. 4(4): p. 637-41.
8. Bratcher, J. M. and B. I. Korelitz, Toxicity of infliximab in the course of treatment of Crohn's disease. Expert Opin Drug Saf, 2006. 5(1): p. 9-16.
9. Scheinfeld, N., Efalizumab: a review of events reported during clinical trials and side effects. Expert Opin Drug Saf, 2006. 5(2): p. 197-209.
10. Weinberg, J. M., et al., Biologic therapy for psoriasis: an update on the tumor necrosis factor inhibitors infliximab, etanercept, and adalimumab, and the T-cell-targeted therapies efalizumab and alefacept. J Drugs Dermatol, 2005. 4(5): p. 544-55.
11. Shear, N. H., Fulfilling an unmet need in psoriasis: do biologicals hold the key to improved tolerability? Drug Saf, 2006. 29(1): p. 49-66.
12. Nissila, M., Use of osmic acid in the topical treatment of exudative synovitis of the knee joint. Scand J Rheumatol Suppl, 1979(29): p. 1-25.
13. Nissila, M., et al., Comparison of chemical, radiation and surgical synovectomy for knee joint synovitis. Scand J Rheumatol, 1978. 7(4): p. 225-8.
14. Menkes, C. J., Is there a place for chemical and radiation synovectomy in rheumatic diseases? Rheumatol Rehabil, 1979. 18(2): p. 65-77.
15. Combe, B., E. Krause, and J. Sany, Treatment of chronic knee synovitis with arthroscopic synovectomy after failure of intraarticular injection of radionuclide. Arthritis Rheum, 1989. 32(1): p. 104.
16. Cruz-Esteban, C. and W. S. Wilke, Innovative treatment approaches for rheumatoid arthritis. Non-surgical synovectomy. Baillieres Clin Rheumatol, 1995. 9(4): p. 787-801.
17. Hilliquin, P., P. Le Devic, and C. J. Menkes, Comparison of the efficacy of nonsurgical synovectomy (synoviorthesis) and joint lavage in knee osteoarthritis with effusions. Rev Rhum Engl Ed, 1996. 63(2): p. 93-102.
18. Bessant, R., et al., Osmic acid revisited: factors that predict a favourable response. Rheumatology (Oxford), 2003. 42(9): p. 1036-43.
19. Hinckley, C. C., Osmium Carbohydrate Complexes, U.S. Pat. NO. 4,346,216, Aug. 24, 1982. 1982.
20. Hinckley, C. C., P. S. Ostenburg, and W. J. Roth, Osmium carbohydrate polymers. Polyhedron, 1982. 1(4): p. 335-8.
21. Hinckley, C. C., S. Sharif, and L. D. Russell, Osmium carbohydrate polymers. Polymeric Materials Science and Engineering, 1983. 49: p. 184-7.
22. Hinckley, C. C., et al., Osmium carbohydrate polymers as potential antiarthritic drugs. ACS Symposium Series, 1983. 209(Platinum, Gold, Othe): p. 421-37.
23. Mirabelli, C. K., et al., Effect of metal containing compounds on superoxide release from phorbol myristate acetate stimulated murine peritoneal macrophages: inhibition by auranofin and spirogermanium. J Rheumatol, 1988. 15(7): p. 1064-9.
24. Elsome, A. M., et al., Antimicrobial activities in vitro and in vivo of transition element complexes containing gold(I) and osmium(VI). J Antimicrob Chemother, 1996. 37(5): p. 911-8.
25. Hinckley, C. C., S. Sharif, and L. D. Russell, Electron microscopic studies of osmium carbohydrate polymers. Met.-Containing Polym. Syst., 1985: p. 183-95.
26. Hinckley, C. C., Osmium based anti-inflammatory agents. Precious Met. Proc. Int. Precious Met. Inst. Conf., 9th, 1986: p. 391-401.
27. Hinckley, C. C., et al., Osmium based antiarthritics: the reaction of osmium-carbohydrate polymers with oxygen. Oxy Radicals Their Scavenger Syst., Proc. Int. Conf. Superoxide Dismutase, 3rd, 1983. 2: p. 388-91.
28. Von Reis, G. V. and A. Svensson, Intra-articular injection of osmic acid in painful joint affections. Acta Med. Scand., 1951. 140: p. 27-32.
29. Kajander, A. and A. Ruotsi, The effect of intra-articular osmic acid on rheumatoid knee joint affections. Ann Med Intern Fenn, 1967. 56(3): p. 87-91.
30. Delcambre, B., et al., [Synoviorthesis with osmic acid in the treatment of rheumatoid disease. Remarks and evaluation of 5 years of its use]. Lille Med, 1976. 21(10): p. 813-20.
31. Martio, J., et al., The effect of intra-articular osmic acid in juvenile rheumatoid arthritis. Scand J Rheumatol, 1972. 1(1): p. 5-8.
32. Ligniere, G. C. and F. Fantini, [Treatment with osmic acid for juvenile rheumatoid arthritis]. Reumatismo, 1973. 25(1): p. 121-4.
33. Boussina, I., et al., Osmium deposits detected by x-ray after synoviorthesis of the knee. Scand J Rheumatol, 1976. 5(1): p. 53-9.
34. Nissila, M., H. Isomaki, and S. Jalava, Reversible renal side effects of intra-articular osmic acid injection. Scand J Rheumatol, 1978. 7(2): p. 79-80.
35. Nissila, M., Absence of increased frequency of degenerative joint changes after osmic acid injections. Scand J Rheumatol, 1978. 7(2): p. 81-4.
36. Sheppeard, H., A. Aldin, and D. J. Ward, Osmic acid versus yttrium-90 in rheumatoid synovitis of the knee. Scand J Rheumatol, 1981. 10(3): p. 234-6.
37. Markovits, D., et al., [Chemical synovectomy in arthritis by intra-articular injection of osmic acid]. Harefuah, 1998. 134(7): p. 520-3, 591.
38. Molho, P., et al., A retrospective study on chemical and radioactive synovectomy in severe haemophilia patients with recurrent haemarthrosis. Haemophilia, 1999. 5(2): p. 115-23.
39. Oka, M., A. Rekonen, and A. Ruotsi, The fate and distribution of intra-articularly injected osmium tetroxide (Os-191). Acta Rheumatol Scand, 1969. 15(1): p. 35-42.
40. Courtney, P. and M. Doherty, Joint aspiration and injection. Best Pract Res Clin Rheumatol, 2005. 19(3): p. 345-69.
41. Kim, S. F., D. A. Huri, and S. H. Snyder, Inducible nitric oxide synthase binds, S-nitrosylates, and activates cyclooxygenase-2. Science (Washington, D.C., United States), 2005. 310(5756): p. 1966-1970.
42. Panzer, U. and M. Uguccioni, Prostaglandin E2 modulates the functional responsiveness of human monocytes to chemokines. Eur J Immunol, 2004. 34(12): p. 3682-9.
43. Nakayama, T., et al., Prostaglandin E2 promotes degranulation-independent release of MCP-1 from mast cells. J Leukoc Biol, 2006. 79(1): p. 95-104.
44. Vestergaard, C., et al., Expression of CCR2 on monocytes and macrophages in chronically inflamed skin in atopic dermatitis and psoriasis. Acta Derm Venereol, 2004. 84(5): p. 353-8.
45. Sedgwick, J. B., P. R. Bergstresser, and E. R. Hurd, Increased superoxide generation by normal granulocytes incubated in sera from patients with psoriasis. The Journal of investigative dermatology., 1981. 76(3): p. 158-63.
46. Er-raki, A., M. Charveron, and J. L. Bonafé, Increased superoxide anion production in dermal fibroblasts of psoriatic patients. Skin pharmacology: the official journal of the Skin Pharmacology Society., 1993. 6(4): p. 253-8.
47. Frank, S., et al., Identification of copper/zinc superoxide dismutase as a nitric oxide-regulated gene in human (HaCaT) keratinocytes: implications for keratinocyte proliferation. Biochem J, 2000. 346 Pt 3: p. 719-28.
48. Quint, P., et al., Crystal structure of nitrated human manganese superoxide dismutase: Mechanism of inactivation. Free Radical Biology & Medicine, 2006. 40(3): p. 453-458.
49. Quijano, C., et al., Reaction of peroxynitrite with Mn-superoxide dismutase. Role of the metal center in decomposition kinetics and nitration. Journal of Biological Chemistry, 2001. 276(15): p. 11631-11638.
50. Goldstein, S., G. Czapski, and A. Heller, Osmium tetroxide, used in the treatment of arthritic joints, is a fast mimic of superoxide dismutase. Free Radic Biol Med, 2005. 38(7): p. 839-45.
51. Anderson, L. H. and D. M. Yost, The properties of osmium tetroxide in carbon tetrachloride solutions. The thermodynamic constants of osmium tetroxide. Journal of the American Chemical Society, 1938. 60: p. 1822-5.
52. Nisel'son, L. A., E. L. Chuvilina, and A. A. Gasanov, Phase equilibrium and the relative volatility of the system OsO4-H2O. Doklady Akademii Nauk, 1995. 344(6): p. 785-786.
53. Hildebrand, J. H., Several solutions of nonpolar substances. Journal of Physical Chemistry, 1939. 43: p. 109-17.
54. Vandendael, Y., Poly(vinylidene chloride). Industrial Polymers Handbook, 2001. 2: p. 931-951.
55. Waris G, Huh K W, Siddiqui A. Mitochondrially associated hepatitis B virus X protein constitutively activates transcription factors STAT-3 and NF-kappa B via oxidative stress. Mol Cell Biol. November 2001; 21(22):7721-30.
56. Sano S. et al., Stat3 links activated keratinocytes and immunocytes required for development of psoriasis in a novel transgenic mouse model. Nat Med. 2005 11(1):43-9.

What is claimed is:

1. A pharmaceutically acceptable topically applied composition containing a compound of a platinum group element for treatment of psoriasis.

2. The composition according to claim 1 where the platinum group element is osmium.

3. The composition according to claim 2, where the nominal valence of the osmium compound is at least four.

4. The composition according to claim 3, where the nominal valence of the osmium compound is at least six.

5. The composition according to claim 4, where the nominal valence of the osmium compound is eight.

6. The composition to claim 2, where at least three of the atoms neighboring the osmium atom of the compound are oxygen atoms or water-exchangeable.

7. The composition according to claim 6, where at least four of the atoms neighboring the osmium atom of the compound are oxygen atoms or water-exchangeable.

8. The composition according to claim 2, where the composition is a water-based matrix containing an osmium compound.

9. The composition according to claim 2, where the composition is an oil- based matrix containing an osmium compound.

10. The composition according to claim 4, where the nominal valence of the osmium compound is six.

11. The composition according to claim 5, where the osmium compound is osmium tetroxide.

12. The composition according to claim 11, where the osmium tetroxide is in the gas phase.

13. The composition according to claim 1 where the compound of the platinum group element catalyzes the dismutation of the superoxide radical anion under physiological conditions.

14. The composition according to claim 15 where the platinum group element is osmium.

15. The composition according to claim 2 where the osmium compound catalyzes the dismutation of the superoxide radical anion under physiological conditions

16. A dressing, adhered to or otherwise applied to the skin, or proximal to the skin, containing an osmium compound for the treatment of psoriasis.

17. The dressing according to claim 17 releasing said osmium compound in a controlled manner.

18. A method of treating psoriasis with a composition containing an osmium compound.

19. The method according to claim 19 where the composition is topically applied.
